(19) United States
(12) Patent Application Publication
(10) Pub. No.: US 2024/0000077 A1
(43) Pub. Date: Jan. 4, 2024

(54) COMPOSITION AND METHOD FOR ENHANCE UVA/UVB RESISTANCE OF AGROCHEMICAL FORMULATION, USE OF THE COMPOSITION, AND AGROCHEMICAL FORMULATION

(71) Applicant: Oxiteno S.A. Indústria e Comércio, São Paulo (BR)

(72) Inventors: Poliana Cardoso GUSTAVSON, Mauá (BR); Mariana MARTINS REIS ANDRADE, Mauá (BR); Fabiana IANHEZ BUZZUTTI, Mauá (BR); Flávia REGIANINI MONTIBELLER, Mauá (BR); Bruna MENDONÇA, Mauá (BR)

(73) Assignee: Oxiteno S.A. Indústria e Comércio, São Paulo, SP (BR)

(21) Appl. No.: 18/039,548

(22) PCT Filed: Nov. 10, 2021
(86) PCT No.: PCT/BR2021/050491

§ 371 Date: May 31, 2023

(30) Foreign Application Priority Data
Dec. 2, 2020 (BR) BR 10 2020 024677

(51) Int. Cl. A01N 43/16; A01N 25/22

(52) U.S. Cl. CPC A01N 43/16; A01N 25/22

(57) ABSTRACT

The present invention refers to a synergistic composition for UVA/UVB protection comprising tannins and additives, being the condensed tannins, hydrolysable tannins or mixtures thereof, as pure or later alkoxylated. The composition can be embodied into agrochemical formulations to enhance the UVA/UVB resistance of these formulations. The UVA/UVB protection granted by the composition of the present invention to the active ingredients of agrochemical formulations improves the performance of products applied in the field, whether they are applied directly on the plant, on the soil or in seed treatment.

### FIELD OF THE INVENTION

The present invention belongs to the field of agrochemical formulations and refers to a synergistic composition comprising tannins and additives capable of providing UVA/UVB protection to these formulations.

### BACKGROUND OF THE INVENTION

The ultraviolet region of the sunlight spectrum (240-400 nm) constitutes a smaller proportion of sunlight reaching the Earth's surface, but the effects on living organisms are disproportionately large. UV photons can be absorbed by a wide range of biomolecules, including nucleic acids (DNA and RNA), proteins and, mainly, low molecular weight metabolites, causing its transition to excited states capable of inducing chemical reactions that result in cell damage. Based on the interaction mechanisms and severity with photosensitive molecules, the solar spectrum can be divided into ultraviolet A (UVA, 315-400 nm) and ultraviolet B (UVB, 280-315 nm). The UVA effects occur through the sensitization of molecules that, upon absorption, interact with oxygen, generating oxygen-reactive species. These, in turn, can oxidize a wide variety of cellular targets. UVB damage, in turn, results from direct absorption by target molecules (DNA and protein) and is independent of the oxygen presence (Gao & Garcia-Pichel (2011) Nature Reviews 9: 791-820).

Microorganisms (fungi, bacteria, viruses) are more or less sensitive to UVA/UVB light depending on its origin and diversification. In other words, if the microorganism lives in the soil, it will likely have less endogenous UV protection (i.e., produced by the organism itself), compared to those whose development takes place on the leaves of a plant. In fact, some soil microorganisms, such as the fungi Isaria fumosorosea and Beauveria bassiana, applied in agriculture as pesticides are highly sensitive to UVA/UVB light and need molecules acting as protectors in its formulation, as it loses viability (survival) and presumable functionality when applied to plants and exposed to sunlight. Likewise, metabolites (e.g., proteins, dsRNA, DNA) and viral particles also show high sensitivity to UVA/UVB and may have its structural conformation changed if subjected to this radiation, losing its effect in the field. Thus, the UVA/UVB protectors use in microbiological formulations is essential to guarantee the performance in the field.

Some conditions must be strictly obeyed for a given molecule to be classified as a UVA/UVB protector: (i) it must absorb light in the length of 240-400 nm (UV) with a high absorption coefficient, and (ii) its concentration in the microorganism should be sufficient to cause a substantial reduction in the UV dose received. It must, of course, not be toxic to microorganisms, and must be anchored in its cellular structure in such a way that the molecule conformation is optimal for intercepting light and acting as a shield. In addition, it must be active during the most sensitive stages of the microorganism's life cycle, such as the inactive phase (spores) and germination—a stage in which it leaves the inactive phase and activates its metabolism, multiplying its cells (Balskus & Walsh (2010)) Science 24: 1653-1656).

Ideally, it should also be demonstrated that the presence of the UVA/UVB protector adds beneficial effects to microbiological formulations, such as an increase in the growth or survival rate, compared to a control group, in which the molecule is not added (Garcia-Pichel (1998) Origins of Life and Evolution of Biospheres 28: 321-347).

There are two main challenges in selecting molecules that act as UVA/UVB protectors for application in microbiological formulations, (i) the biocompatibility with microorganisms, and (ii) the difficulty of suggestive formulations keeping the UVA/UVB protector anchored to the microorganisms to be, in fact, effective.

By “biocompatibility” is meant the property of biological units (cells, tissues, organisms) to react to the presence of non-living materials. In other words, the beneficial or toxic influence of non-living compounds (including chemicals) on living beings. In this document this concept is applied to evaluate the influence of additives containing UVA/UVB protector on the viability of fungi and bacteria (Cardoso-Gustavson et al. (2020) ASTM International Standards Organization).

UVA/UVB protectors used in agrochemical formulations for photosensitive chemical actives or applied in cosmetic formulations are commonly para-amino benzoic acid, cinnamates, salicylates, bemotrizinol (Tinosorb S®, BASF), avobenzone (Neo Heliopan357®, Symrise), cctyltriazone (Uvinul T 150®, BASF), enzacamene (New Heliopan MBC®, Symrise), oxybenzone (Newo Heliopan BB®, Symrise), padimate O (Escalol 507®, Ashland), octonoxate (Escalol 557®, Ashland), Octisalate (Escalol 587®, Ashland) However, chemical protectants do not have a chemical structure presumably compatible with microorganisms (Lozano et al. (2020) Science of the Total Environment 722: 137803, BR 112019013816, ES 2638713, U.S. Pat. No. 8,404,263, US 2012/0052187, US 2011/0257265, US 2011/0237655, EP 0057160), and many of them have serious environmental toxicity problems (Buser et al. (2006) Environmental Science and Technology 40: 1427-1431).

Some formulations containing inocula mention a composition that normally comprises a mixture of various UVA/UVB protectors, such as aromatic amino acids and lignosulfonates, in addition to carotenoids, cinnamates, melanins, mycosporins and salicylates (BR112019013816A2, ES2638713A1, U.S. Pat. No. 8,404,263). This is due to the complexity of protecting microorganisms for its intrinsic characteristics, and the inefficiency of these components as protectors when isolated.

Regarding the difficulty in keeping the protective molecules anchored to the cells, it can mention the so-called physical protectors, such as zinc oxide (ZnO) and titanium dioxide (TiO₂). Although they are efficient in scattering light, it is not possible to adhere to the structure of the microorganism, so that its proximity to the microorganisms' cells becomes random once the formulation is applied in the field. Thus, the protection efficiency is very low or non-existent.

Phenolic compounds are known to absorb light in the UVA/UVB spectrum. In particular, the flavonoid class has been applied as UV protectors in the cosmetic industry. However, these molecules usually have high interaction with cells, and offer germicidal action, and its use in microbiological formulations is not convenient. In addition, stabilization of the phenolic extract normally occurs through ethanol and other organic solvents, which enhances the germicidal action of these products (Travasarou et al. (2019) Cosmetics 6:1-7).

Phenolic compounds are robust antioxidants and provide photoprotection. However, a large part (phenolic acids, flavonoids) is not industrially applied due to the difficulties caused by the photo instability of these protectors. However, some highly complex polyphenols, such as tannins and lignin, are stable and have been used industrially in the most different applications.

Tannins are reported as reinforcing components for polypropylene with anti-UV properties (Liao et al. (2019) Polymers, 11: 102-117), or in the production of polyurethanes and cosmetic applications, with no anti-UV protection being invoked (BR 112015031873). Lignin, on the other hand, stands out among the polyphenols currently positioned as sunscreen in agrochemical formulations, described as an encapsulating agent (microcapsules) for actives sensitive to UV radiation, as described in patents EP 1993/0653158 and US 1994/5529772. However, it is noteworthy that the use of this molecule in encapsulation is done in combination with other materials, because, like tannins, lignin also has high antimicrobial activity.

Tannins constitute a group of complex polyphenols with high molecular weight (between 500-3000 Da). In complexes with polysaccharides, alkaloids and proteins, this molecular weight can increase to up to 20000 Da, having reactions features of phenols. The chemical structure of tannins depends on the plant species from which they are extracted. Currently, more than 8000 different tannins have been isolated and chemically characterized. They are characterized by being highly potent antioxidants, which act directly on the interception of UVA/UVB light, in addition to providing protection against damage caused by free radicals after exposure to UV light (Brillouet et al. (2013) Annals of Botany 112: 1003-1014).

All tannins have common characteristics, which allows the classification of this type of compounds into two main groups, hydrolyzable or condensed tannins. The hydrolyzable tannins (gallotannins, ellagitannins and complex tannins) are derived from a reaction with glucose. At the center of a hydrolyzable tannin there is a carbohydrate such as d-glucose. The hydroxyl groups from carbohydrates are partially or fully esterified with phenolic acids, such as gallic acid (in gallotannins). Hydrolyzable tannins can be hydrolyzed by weak acids or weak bases to produce carbohydrates and phenolic acids. Condensed tannins, also called proanthocyanidins, are 2-50 (or more) flavonoid units (catechins) polymers, which are not susceptible to hydrolysis (Saslowsky & Winkel-Shirley (2001) The Plant Journal 27:37-48).

Hydrolyzable and condensed tannins have strong antibacterial, antifungal and antiviral action. Typically, tannins are known for its ability to precipitate proteins, pectins and cellulose, acting as enzyme inhibitors. The literature shows the use of tannins as antimicrobial compounds and even as a nematicide (US 2020/10683244, US 2020/0296965), which deals precisely with the use of tannins in agrochemical formulations as a bactericide and fungicide. In fact, the inhibitory action of tannins on the growth of bacteria, fungi, yeasts and viruses has been very well documented since the 1950s (Scalbert (1991) Phytochemistry 30: 3875-3883).

Therefore, the use of condensed or hydrolyzable tannins or a mixture thereof as a UV protector in agrochemical formulations containing microorganisms that must be kept alive after field application is not obvious or intuitive—on the contrary, such a combination would not be prudent.

Since the tannins are natural origin compounds, there are no problems regarding its degradation in soil or water. In fact, tannins have no harmful effects on the environment, especially tannins in raw way.

In fact, due to the chemical structure of tannins, they easily form complexes with various polyvalent ions, such as iron, manganese and zinc, which are of special agronomic interest as fertilizers (EP 1097912, EP 1803351). Furthermore, interactions between tannins and the chemistry of the nitrogen forms used as fertilizers enhance functionality in plants when applied to the soil (Rao et al. (2000). Plant Molecular Biology 44: 345-358). In fact, tannins can act as nitrogen release modulators and iron complexing agent. Furthermore, tannins are also applied as soil bio-remediators precisely because of its high capacity to form complexes with metals, including heavy metals such as Cr⁶⁺, Cd²⁺, and also Cu²⁺ and Fe²⁺ (Prigione et al. (2018). Applied Microbiology and Biotechnology 102, 4203-4216).

Although the use of tannins as complexing agents in soil is known, there are no references or mention to the application of these polyphenols as an UVA/UVB radiation protector of soil or leave fertilizers. Mainly in leave fertilizers, these rays cause photodegradation of the fertilizers, impairing the performance of these products. In this sense, the application of tannins (condensed, hydrolysable) with the purpose of UVA/UVB protecting in leave fertilizers is also unprecedented.

The same photodegradation sensitivity reasoning can be applied to biostimulants.

The use of tannins as biostimulants is due to its ability, when degraded by the soil microbiota, to act as a fertilizer (US 2016/0023960), or as a nitrification inhibitor, acting in synergy with bacteria (US 2012/8211200). So far, there is no mention to the use of tannins as UVA/UVB protectors for formulations containing biostimulants.

### BRIEF DESCRIPTION OF THE FIGURES

FIG. 1: composition samples comprising tannins and different additives.

### DETAILED DESCRIPTION OF THE INVENTION

The present invention refers to a composition for the UVA/UVB protection comprising tannins and additives, as well as agrochemical formulations containing said composition.

Tannins can be condensed, hydrolysable or mixtures thereof, in its raw form or subsequently alkoxylated. Both the condensed and hydrolysable tannins used in the present invention are commercially available.

Preferably, the hydrolyzable tannins of the present invention consist of a central glucose molecule linked to gallic acid (gallotannins) or hexahydroxy diphenic acid (ellagitannins) molecules. Condensed tannins consist of oligomers and polymers of polyhydroxyflavan-3-ol monomeric units linked by acid-labile bonds 4→6 or 4→8. These polymers have irregular, often branched chains, and generally contain more than one type of basic unit of setting.

Alkoxylated tannins can be obtained by reacting the tannins in its raw form with an alkylene oxide selected from the group consisting of ethylene oxide, propylene oxide or butylene oxide, preferably ethylene oxide. Alkoxylation can be performed on hydroxyls naturally present in hydrolyzable or condensed tannins, solubilized in different polar or nonpolar solvents, respectively. Different alkoxylation methods can be used. For use in the present invention, the alkoxylated tannins must contain at least 20 moles of the alkylene oxide employed, preferably 20 to 40 moles of ethylene oxide. The greater the alkoxylation degree, the greater the biocompatibility of the final material obtained. Among the advantages of using alkoxylated tannins are the reduction—and possible removal—of toxicity in water and the improvement of dispersibility.

The additives employed in the composition of the present invention are selected from polyethylene glycol, ethoxylated phenol and/or pure ethoxylated glycol, which form a synergistic mixture with the tannins. These additives are able to bind to tannin molecules and avoid direct interaction between these molecules and the microorganisms present in the agrochemical formulations in which the composition will be incorporated. Thus, when added to agrochemical formulations containing microorganisms, tannins form a stable complex that has UVA/UVB protective activity as an integral part of its structure.

Regarding usage, the composition comprising tannins and additives of this invention may be applied directly to a fermentation broth of microorganisms, or be embodied into an agrochemical formulation containing microorganisms, fertilizers or biostimulants, or a combination thereof, said formulation as a concentrated suspension, wettable powder, dispersible granules, oil dispersion, emulsifiable concentrate, emulsion or microemulsion, among others, and benefit the performance of these formulations when applied to seeds, leaves or soil.

In the present invention, by “microbiological formulations” or “agrochemical formulations containing microorganisms” is meant compositions containing fungi, bacteria, viral particles, macromolecules or particles of different biological nature (proteins, polysaccharides, lipids and combinations thereof) produced in an extracellular medium by microorganisms, proteins, DNA, RNAi acting as pesticides.

As an example, we can mention the main microorganisms currently formulated, Amblyseius sp., Azospirillum sp., Bacillus sp., Baculovirus sp., Beauveria sp., Cotesia sp., Cryptolaemus sp., Deladenus sp., Heterorhabditis sp., Isaria sp., Metarhizium sp., Neoseiulus sp., Orius sp., Paecilomyces sp., Pasteuria sp., Phytoseiulus sp., Pseudomonas sp., Stratiolaelaps sp., Telenomus sp., Trichoderma sp., Trichogramma sp., or mixtures thereof.

By “biostimulants” is meant compositions containing plant activators and/or growth regulators, for example from the chemical group: dioxocyclohexanecarboxylic acid, indole alkanoic acid, aliphatic alcohol, quaternary ammonium, carbimide, carboxanilide, cycloalkene, cyclohexadione, cytokinin, dinitroaniline, ethylene inhibitor, ethylene precursor, gibberellin, pyridazinedione, sesquiterpenes, triazole, benzothiadiazole, or mixtures thereof. These compositions may also include signaling molecules such as nitric oxide, methyl salicylate, methyl jasmonate, proteins (and thus enzymes), polypeptides, polyamines, and complex compositions such as algae extracts, fulvic acid, humic acid, and microorganisms such as PGPR (plant growth promoting rhizobacteria), bacteria and fungi producing compounds that stimulate plant growth and reduce the deleterious effects of abiotic stresses such as water stress.

By “fertilizer formulations” is meant compositions containing macronutrients (N, P, K, S, Ca, Mg or mixtures thereof) and/or micronutrients (Fe, Zn, Mn, Cu, Ni, Cl, Mo, B, Si, If, Al, Co, V, Na or mixtures thereof).

Thereafter, the invention is described as examples, which illustrate the obtainment of the composition for UVA/UVB protection comprising tannins and additives, properties of the composition obtained and its embodiment into other formulations.

### Example 1—Method for Obtaining the Composition Comprising Tannins and Additives and Method to Enhance the UVA/UVB Resistance of Agrochemical Formulations

Condensed tannins, hydrolysable tannins or mixtures thereof are added to at least one additive of polyethylene glycol, ethoxylated phenol and/or pure ethoxylated glycol, under stirring, in sufficient amount to obtain a tannin concentration of 0.02 to 20%, preferably between 0.2 and 10% in weight of the composition. The same procedure is employed when alkoxylated tannins are used.

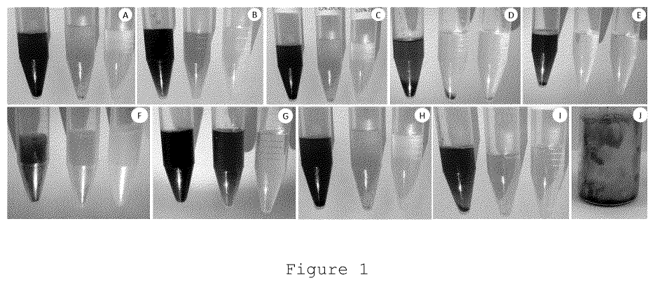

FIG. 1 shows the aspects of tannin composition samples with different additives. (A-I) Tannins at concentrations of 2%, 0.2% and 0.02% (from left to right) in concentrated additives, respectively. (A) Solubility in water. (B) Solubility in polyol. (C) Suspensibility in ethoxylated glycol. (D) Suspensibility in sorbitol ester. (E) Solubility in ethoxylated sorbitol ester. (F) Suspensibility in ethoxylated alcohol. (G) Solubility in propoxylated ethoxylated alcohol. (H) Suspensibility in polyethylene glycol. (I) Solubility in ethoxylated phenol. (J) Suspensibility of 2% tannin in oxyalkene copolymer. The same composition obtained can be added/embodied at a concentration of 0.01 to 5%, preferably between 1 and 3%, to an agrochemical formulation containing solvents, emulsifiers, dispersants, moisturizing and other components suitable for the purposes of said formulation. Also, the composition of tannins and additives, totaling between 0.02 to 30%, preferably between 0.02 and 5% of the final agrochemical formulation, can be added to a broth fermentation containing microorganisms or microorganisms in solid agrochemical formulations. Also, the composition of tannins and additives, totaling between 0.02 and 30%, preferably between 0.02 and 5% of the final agrochemical formulation, can be added to a fermentation broth containing microorganisms or microorganisms in solid agrochemical formulations. Thus, using the composition of the present invention, the method to enhance the UVA/UVB resistance of agrochemical formulation comprises the steps of: adding the condensed tannins, hydrolysable tannins or its mixtures to at least one additive, under stirring, in a sufficient amount to obtain a tannin concentration from 0.02 to 20% in weight of composition; embodiment of said composition to an agrochemical formulation; and adding the composition to a liquid agrochemical formulation containing fermentation broth of microorganisms or microorganisms into solid formulations. The addition of tannins to additives, already described, can be done by solubilization or dispersion. According to the method of this invention, agrochemical formulations may contain at least one microorganism in fermentation broth or powdered spores, solvents, moisturizing agents, dispersing agents and emulsifying agents, and may further comprise fertilizers or biostimulants.

### Example 2—Evaluation of Biocompatibility and UVA/UVB Protection Granted by the Composition Containing Condensed Tannins and Additives to Bacteria

In this example, Bacillus subtilis was employed as a model of gram-positive bacteria, and microorganisms were added to culture media containing different samples of the composition from Example 1. Biocompatibility is expressed as the survival count of microorganisms compared to the control (microorganisms cultivated in a culture medium with no products added). Survival may be lower than the control, characterizing the product as incompatible (thus, its use is not prudent), similar to or better than the control, characterizing it as compatible. The treatment is also performed with components without the composition containing tannins and additives, in order to identify synergistic or antagonistic actions or indifferent actions between the composition and the microorganisms.

After this selection, the compositions were then evaluated for potential UVA/UVB protection given to microorganisms. There may be an interaction between tannins and a certain component that cancels out the protective effect given by the polyphenol. Conversely, a given component can enhance the effect of tannins, either by mediating a position of the molecule that favors the interception of UVA/UVB rays, or by also having a chemical structure that amplifies the dissipation of excess energy promoted by these rays. The evaluation of the UVA/UVB protection potential is performed by exposing culture medium plates containing different samples of composition and microorganisms to UVA/UVB rays inside a growth chamber for 210 minutes after the inoculation of the microorganisms, that is, after applying microorganisms in the Petri dish (Dias et al. (2018) Fungal Biology 122: 592-601). According to the literature, this initial period of microorganism development in dish is the most critical and sensitive to UVA/UVB rays (Dias et al. (2019) Fungal Biology 124: 263-272). The results of the UVA/UVB protective activity of the composition samples containing condensed tannins and additives on the development of Bacillus subtilis are shown in Table 1.

TABLE 1
Biocompatibility and UVA/UVB protective
activity of composition samples comprising condensed tannins
and additives.

| | Control | | UVA/UVB 210 min | |
|---|---|---|---|---|
| | | positive | | positive |
| | sample | control | Sample | control |
| Component | (CFU/ml) | (CFU/ml) | (CFU/ml) | (CFU/ml) |
| Water | 0.00E+00 | 1.00E+03 | 0.00E+00 | 1.00E+03 |
| 2% condensed | 1.00E+04 | 1.00E+03 | 1.00E+02 | 1.00E+01 |
| tannins in | | | | |
| polyethylene | | | | |
| glycol | | | | |
| 2% condensed | 1.00E+04 | 1.00E+03 | 1.00E+01 | 0.00E+00 |
| tannins in | | | | |
| alkoxylated | | | | |
| phenol | | | | |
| 2% tannins in | 1.00E+03 | 1.00E+03 | 1.00E+03 | 1.00E+01 |
| alkoxylated | | | | |
| glycol | | | | |

Biocompatibility is checked by counting colony-forming units of Bacillus subtilis in culture media containing the different samples of the composition and bacteria in culture medium with no composition added (positive control), and the UVA/UVB protective activity subjecting reciprocal samples to UV rays during the period of multiplication of microorganisms. Table 1 shows the incompatibility between the condensed tannins solubilized in water, reinforcing the antimicrobial nature of this polyphenol. In addition, the tests show some of the chemical additives in which the condensed tannins act synergistically as UVA/UVB protectors for microorganisms, showing CFU values higher than the control subjected to the same condition.

### Example 3—Agrochemical Formulations Containing the Composition of Tannins and Additives

The present invention also describes formulations containing the composition of tannins and additives, and the formulations can be applied to the soil, directly or through irrigation system by gravity, sprinkler, drip, infiltration, central irrigation pivot, or submersion; directly on the plants, by means of sprinkling; or through seed treatment.

The microbiological formulations to which this invention refers comprise from 1-80% fermentation broth (v:v) or powdered spores (p:p), preferably from 20-99% (v:v or p:p) of components chemicals that work to improve the physicochemical parameters of the formulation (wettability, suspendability, spreadability, emulsifiers, components that promote adhesion and retention), water or inert sufficient for (a.s.p.) 1 L or 1 kg.

In a preferred embodiment, the microbiological formulation comprises 1-80% (v:v or m:m) fermentation broth or bacterial or fungal cell spores powder, 1-40% glycol, ethoxylated glycol, ethoxylated and propoxylated glycol, and/or polyethylene glycol for microorganism encapsulation, 1-20% (v:v or m:m) of moisturizing agents, such as ethoxylated alcohols, ethoxylated and propoxylated alcohols, co-polymers, sorbitan derivatives, or mixtures thereof, 1-20% (v:v or m:m) of dispersants, such as glycols and polyglycols, polymers, ethoxylated alkylphenols, phosphated, sulfated, ethoxylated/propoxylated components, acrylates or mixtures thereof, 1-20% (v:v or m:m) of emulsifiers, such as ethoxylated oils, polysorbates and sorbitan esters, or mixtures thereof, 0.2-5% (v:v or m:m) of the composition containing tannins described in this invention, 1% (v:v or m:m) of other components (antifreeze, antioxidants, antimicrobials) and inert q.s.p. 1 L or 1 kg.

In another preferred embodiment, the microbiological formulation comprises 1-80% (v:v or m:m) fermentation broth or powdered bacterial or fungal cell spores, 1-40% (v:v or m:m) solvents, such as plant or mineral origin oils or esters, or mixtures thereof for encapsulating the microorganism, 1-20% (v:v or m:m) of moisturizing agents, such as ethoxylated alcohols, ethoxylated and propoxylated alcohols, co-polymers, derivatives of sorbitan or mixtures thereof, 1-20% (v:v or m:m) of dispersants, such as glycols and polyglycols, polymers, ethoxylated alkylphenols, phosphated, sulfated, ethoxylated/propoxylated components, acrylates or mixtures thereof, 1-20% (v:v or m:m) of emulsifiers, such as ethoxylated oils, polysorbates and sorbitan esters, or mixtures thereof, 0.2-5% (v:v or m:m) of the composition containing tannins and additives described in this invention, 1% (v:v or m:m) of other components (antifreeze, antioxidants, antimicrobials) and inert q.s.p. 1 L or 1 kg.

To validate the composition of this invention for UVA/UVB protection in a microbiological formulation as being suitable for maintaining the viability of microorganisms, the number of colony forming units (CFU) must be counted. This measure must be taken immediately after the fermentation of the microorganism, without the application of any formulation (control), and taken periodically in order to assess the drop in viability of the microorganism over time, that is, the shelf life of the formulation.

The different samples of the composition must be equally evaluated in order to show the additives' ability to keep the viability count similar (or with a significantly lower reduction) to that obtained in the control taken immediately after fermentation.

Namely, the CFU is a unit of measurement used to estimate the number of viable bacteria or fungi, that is, capable of multiplying by binary fission under controlled conditions.

Table 2 shows the CFU count of microorganisms not formulated after fermentation (control) and formulated with composition samples containing condensed tannins and additives, and evaluated after a certain time.

TABLE 2
Count of colony forming units in
microbiological formulations containing composition samples
for UVA/UVB protection described in Table 1.

| Formulation | Microorganism | Count (CFU) |
|---|---|---|
| fermentation broth | Bacillus subtilis | 1.7 × 10⁹ (newly |
| with no formulation | (bacteria) | fermented*) |
| (control) | | |
| glycol base | Bacillus subtilis | 1.6 x 10⁹ (after 1 year |
| suspension | (bacteria) | at room temperature) |
| concentrate suspension | | |
| containing UV | | |
| protector | | |
| oil-based suspension | Bacillus subtilis | 7.5 × 10⁸ (after 1 year |
| concentrate containing | (bacteria) | at room temperature) |
| UV protector | | |
| fermentation broth | Bacillus | 1.9 × 10¹⁰ (newly |
| with no formulation | amyloliquefaciens | fermented*) |
| (control) | (bacteria) | |
| glycol base | Bacillus | 1.6 × 10⁹ (after 1 year |
| concentrated | amyloliquefaciens | at room temperature) |
| suspension containing | (bacteria) | |
| UV protector | | |
| oil-based concentrated | Bacillus | 5 × 10⁹ (after 1 year |
| suspension containing | amyloliquefaciens | at room temperature) |
| UV protector | (bacteria) | |
| spores with no | Trichoderma | 2.4 × 10¹⁰ (after 3 |
| formulation (control) | harzianium | months at room |
| | (fungi) | temperature) |
| oil-based dispersible | Trichoderma | 1.22 × 10¹⁰ (after 3 |
| granule containing UV | harzianium | months at room |
| protector | (fungi) | temperature) |

*count performed after the microorganism has been fermented. The broth completely loses its viability (i.e., the amount of colony-forming units is zero) at room temperature when not formulated.

Therefore, the UVA/UVB protection given by the composition comprising tannins and additives to microorganisms is then showed.

1. A composition to enhance UVA/UVB resistance of agrochemical formulation, wherein the composition comprises hydrolyzable, condensed tannins or mixtures thereof, and an additive, wherein:
the hydrolysable tannins consist of a central glucose molecule linked to gallic acid or hexahydroxy diphenic acid molecules;
the condensed tannins consist of oligomers and polymers of polyhydroxyflavan-3-ol monomeric units linked by acid-labile bonds 4→6 or 4→8; and
the additives are selected from polyethylene glycol,
alkoxylated phenol and/or pure alkoxylated glycol, which form a synergistic mixture with the tannins; and
the concentration of tannins is from 0.02 to 20% by weight of the composition.

2. The composition according to claim 1, characterized in that the hydrolyzable, condensed tannins or a mixture thereof are alkoxylated by reaction with an alkylene oxide selected from the group consisting of ethylene oxide, propylene oxide or butylene oxide.

3. The composition according to claim 1, characterized in that the tannins are alkoxylated with at least 20 moles ethylene oxide.

4. Use of the composition according to claim 1, characterized in that it the composition containing tannins as UVA/UVB protector of microorganisms is incorporated into an agrochemical formulation containing microorganisms, fertilizers or biostimulants, or a combination thereof, said formulation being as a concentrated suspension, wettable powder, dispersible granules, oil dispersion, emulsifiable concentrate, emulsion or microemulsion.

5. A method to enhance the UVA/UVB resistance of agrochemical formulation employing the composition according to claim 1, comprising the steps of:
adding hydrolysable tannins, condensed tannins or mixtures thereof to at least one additive, under stirring, in sufficient amount to obtain a tannins concentration from 0.02 to 20% in weight composition;
embodying said composition to an agrochemical formulation;
adding composition to a liquid agrochemical formulation containing a microorganisms fermentation broth or microorganisms into solid formulations;
wherein the tannins are solubilized or dispersed in additives selected from polyethylene glycol, alkoxylated phenol and/or pure alkoxylated glycol, which form a synergistic mixture with the tannins.

6. (canceled)

7. The method according to claim 5, characterized in that the agrochemical formulations contain at least one microorganism in broth fermentation or powdered spores, solvents, moisturizing agents, dispersing agents and emulsifying agents, and it may further comprise fertilizers or biostimulants.

8. An agrochemical formulation to be used in the method according to claim 5, characterized in that it is a microbiological formulation comprising 1-80% (v:v or p:p) fermentation broth or powdered spores of bacterial or fungal cells, or a combination thereof, and (ii) 0.02-5% (v:v or m:m) a composition containing hydrolyzable, condensed tannins or mixtures thereof and an additive, wherein:
the hydrolyzable tannins consist of a central glucose molecule linked to gallic acid or hexahydroxy diphenic acid molecules;
the condensed tannins consist of oligomers and polymers of polyhydroxyflavan-3-ol monomeric units linked by acid-labile bonds 4→6 or 4→8; and
the additives are selected from polyethylene glycol, alkoxylated phenol and/or pure alkoxylated glycol, which form a synergistic mixture with the tannins.

9. The agrochemical formulation according to claim 8, characterized in that the formulation is as water dispersible granules and comprises solvents, such as 1-40% (v:v or p:p) plant or mineral origin, pure or mixed oils or esters, or glycol, ethoxylated glycol, ethoxylated and propoxylated glycol, and/or polyethylene glycol or mixtures thereof.

10. The agrochemical formulation according to claim 9, comprising moisturizing agents, such as 1-20% (v:v or p:p) ethoxylated alcohols, ethoxylated and propoxylated alcohols, co-polymers, sorbitan derivatives, or mixtures thereof.

11. The agrochemical formulation according to claim 9, comprising dispersing agents, being 1-20% (v:v or p:p) glycols, polyglycols, polymers, ethoxylated alkylphenols, phosphate, sulfated, ethoxylated/propoxylated components, and acrylates, or mixtures thereof.

12. The agrochemical formulation according to claim 9, comprising emulsifying agents, such as 1-20% (v:v or p:p) ethoxylated polyols, ethoxylated and propoxylated polyols, sorbitan esters, ethoxylated sorbitan esters, ethoxylated and propoxylated sorbitan esters, ethoxylated oils, ethoxylated alcohols, or mixtures thereof.

13. The agrochemical formulation according to claim 12, comprising 1% (v:v or p:p) of antifreeze, antioxidants, antimicrobials and inert q.s.p. 1 L or 1 kg.

14. The agrochemical formulation according to claim 8, characterized in that it is applied to the soil directly or through a gravity irrigation system, sprinkler, dripping, infiltration, central irrigation pivot, or submersion; directly on the plants by means of sprinkling; or by seed treatment.
